(19) United States
(12) Patent Application Publication
(10) Pub. No.: US 2026/0297663 A1
(43) Pub. Date: Oct. 1, 2026

(54) MULTI-INSTRUMENT DPCR WITH GROUPED SAMPLE ANALYSIS FOR HIGH THROUGHPUT APPLICATIONS

(71) Applicant: LIFE TECHNOLOGIES CORPORATION, Carlsbad, CA (US)

(72) Inventors: Saradwata SARKAR, Newark, CA (US); Alexander Louis ROEMER, Rockford, MI (US); Alan Edgel BARBER, II, Idaho Falls, ID (US); Marissa Kim LEE, San Bruno, CA (US); Noah ELDER, Lafayette, CA (US); Christopher Brandon THOMAS, San Mateo, CA (US); Roger DETTLOFF, Palo Alto, CA (US)

(21) Appl. No.: 19/475,725

(22) PCT Filed: Apr. 17, 2024
(86) PCT No.: PCT/US2024/025023

§ 371 Date: Oct. 15, 2025

Related U.S. Application Data
(60) Provisional application No. 63/460,885, filed on Apr. 20, 2023.

(51) Int. Cl. C12Q 1/686; G16B 25/20; G16B 40/10; G16B 45/00

(52) U.S. Cl. CPC C12Q 1/686; G16B 25/20; G16B 40/10; G16B 45/00

(57) ABSTRACT

Various embodiments for improved dPCR systems and methods are disclosed. Some embodiments provide a multi-instrument dPCR system that facilitates analyzing samples together across multiple arrays of partitions, multiple sample plates, and/or multiple instruments to provide a better and higher throughput dPCR system. Aspects of various embodiments provide one or more of the following: Correction of illumination bias; quality control processing; flexible grouping of samples across partitions, sample plates, and instruments for thresholding and analysis; inter-instrument signal equalization for each dye channel to facilitate grouping samples across instruments; and improved auto-thresholding.

### CROSS-REFERENCE TO RELATED APPLICATIONS

This application claims the benefit of U.S. Provisional Application No. 63/460,885 filed Apr. 20, 2023. This application also has some subject matter relationship to commonly assigned Provisional Application Nos. 63/460,882; 63/460,884;63/460,879, all filed on Apr. 20, 2023. The contents of these applications are incorporated herein by reference in their entirety.

### BACKGROUND

This disclosure generally relates to digital polymerase chain reaction (dPCR) technology.

dPCR typically involves partitioning a PCR reaction mixture including a sample into many thousands of individual partitions (e.g., in individual microchambers) such that each partition contains preferably either no molecules or one molecule (or, less preferably, slightly more than one molecule) corresponding to a target analyte; subjecting the samples to thermocycling in accordance with a PCR assay; measuring fluorescent signals from each partition at an endpoint of the PCR assay; determining a threshold to separate signals for that indicate “positive” result (amplification occurred, target was present) at a corresponding partition from those that indicate a “negative” result (amplification did not occur, target not present) at a corresponding location; and using the binary results for each partition to compute the quantity (concentration) of a target analyte in the sample. The “digital” in the name digital PCR reflects the binary nature of the partition level results used to compute target quantities for the sample as a whole.

Among other things, dPCR quantification is more adept than other PCR methods (e.g., quantitative PCR—also known as “qPCR” or “real-time PCR”) at detecting and quantifying concentrations of hard to detect rare targets, providing a more precise quantitation of samples or analysis (e.g., nucleotide sequences), and measuring low fold changes in analyte concentration. Consequently, digital quantification has many applications in basic research, clinical diagnostics, and environmental testing. For example, digital PCR has been applied to pathogen detection and cancer monitoring, copy number variation analysis, single gene expression analysis, rare sequence detection, gene expression profiling and single-cell analysis, detection of DNA contaminants in bioprocessing, validation of gene edits, and detection of specific methylation changes in DNA as biomarkers of cancer.

Digital quantification can be performed on biological samples that contain or are suspected to contain a target analyte of interest, such as a cell, tissue, or specimen such as hair, a biological fluid such as blood, urine, saliva, etc., a cell cluster such as a microbial colony, or an organism, cell, microbe, bacterium, virus, protein, antibody, or nucleic acids such as such as DNA or RNA molecules. Target analytes include “original” analytes that were originally present in the biological sample as well any “synthetic” analytes that are indicative of the presence of original analytes which may be added or generated during detection, including PCR amplicons, antigen-antibody complexes, etc. In a typical dPCR assay, the sample is partitioned into a large number of smaller test samples, which will ideally contain either one molecule of the target analyte or no molecules of the target analyte such that a separate detection reaction can be carried out in each partition individually. Suitable partitions are individual targets that are sufficiently distanced from other individual targets to allow for individual detection or quantification, which may or may not be fluidically isolated from each other. Partitions may or may not include separating barriers such as walls or membranes or liquids that are immiscible with the sample, or semisolid media. Exemplary partitions include individually distanced targets, e.g., deposited on a substrate such as a glass slide, a tube, open or closed well, droplet, vesicle, chamber or bead, or any representation of an individual signal derived from a target that is distinguishable over background or noise, for example a bright spot over a darker background in a digital or analog image. In digital PCR methods, when the samples are thermally cycled using a PCR apparatus, the samples containing the target concentration are amplified and produce a positive detection signal, while the samples that do not contain the target concentration are not amplified and produce no detection signal. After multiple PCR amplification cycles, the samples are imaged and analyzed for fluorescence, which is used to quantify the target concentration in the samples.

### SUMMARY

Existing dPCR quantification technologies are typically designed around a single plate, single instrument workflow. This imposes limitations on high-throughput biopharmaceutical applications that benefit from large sample volume testing in the process of searching for and developing new therapeutic treatments for diseases. Being able to pool data from multiple samples is especially important in researching rare alleles in order to increase reliability in the results. Also, in large studies covering many patient samples, pooling data for group analysis can increase the reliability of the statistical determination needed to advance medical technologies, especially in biopharmaceutical areas such as oncology and other areas. These needs are not met sufficiently by existing dPCR technology. Embodiments of the present disclosure improve dPCR technology to address these unmet needs.

Some embodiments of the present disclosure address these issues new by providing a multi-instrument dPCR system that facilitates analyzing samples together across multiple arrays of partitions, multiple sample plates, and even multiple instruments to provide a better and higher throughput dPCR system. Aspects of various embodiments provide one or more of the following: Improved correction of illumination bias; improved quality control processing; new and flexible grouping of samples across partitions, sample plates, and instruments for thresholding and analysis; inter-instrument signal equalization for each dye channel to facilitate grouping samples across instruments; and improved auto-thresholding.

Details of these and other embodiments are more fully disclosed with reference to the following drawings.

### BRIEF DESCRIPTION OF THE DRAWINGS

FIG. 1 illustrates a multi-instrument dPCR test and analysis system in accordance with an embodiment of the present disclosure.

FIG. 2 illustrates a sample plate for use with the dPCR test and analysis system shown in FIG. 1.

FIG. 3 is a high-level block diagram illustrating processing carried out by a dPCR analysis system in accordance with some embodiments of the present disclosure.

FIG. 4 is a flow diagram showing the processing steps of inter-instrument equalization processing in accordance with some embodiments of the present disclosure.

FIG. 5 is a flow diagram showing a method for obtaining brightness coefficients for use in inter-instrument signal equalization in accordance with some embodiments of the present disclosure.

FIGS. 6A and 6B are data plots illustrating an example of the effects of applying inter-instrument signal equalization processing in accordance with some embodiments of the present disclosure.

FIG. 7 illustrates a graphical user interface in accordance with some embodiments of the present disclosure.

FIG. 8 illustrates another graphical user interface in accordance with some embodiments of the present disclosure.

FIG. 9 is a flow diagram illustrating processing used to determine a threshold to set for use in dPCR analysis and in a graphical user display associated with such analysis.

FIG. 10 illustrates an exemplary computer system configurable by a computer program product to implement embodiments of the present disclosure.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices.

Accordingly, any of the various embodiments herein may take the form of an entirely hardware embodiment, an entirely software embodiment or an embodiment combining software and hardware aspects. The following specification is, therefore, not to be taken in a limiting sense.

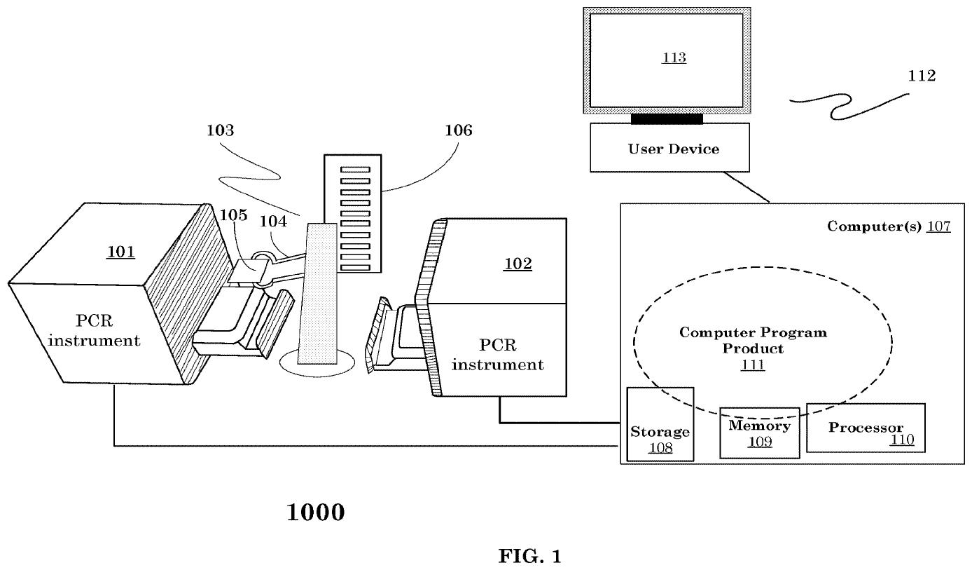

FIG. 1 illustrates a multi-instrument digital polymerase chain (dPCR) reaction test and analysis system 1000 in accordance with an embodiment of the present disclosure. System 1000 comprises first PCR instrument 101, second PCR instrument 102, robotic device 103, sample plate holder 106, sample plates 105, and one or more computers 107 which are communicatively to PCR instruments 101 and 102 and to one or more end user devices 112.

System 1000 is configured to process multiple sample plates 105 at a time without requiring user intervention. For example, robotic device 103, which includes robotic arms 104, can operate to load one sample plate 105 into PCR instrument 101 and another sample plate 105 into PCR instrument 102, and PCR thermal cycling can proceed to process and image both sample plates 105 in parallel. After a round of PCR thermal cycling (which may include 40 cycles, 50 cycles, or some other number), robotic device 103 can remove processed plates 105 from each instrument and then load additional plates 105 from sample plate container 106 into PCR instruments 101 and 102 for PCR processing without requiring human intervention.

Computer-executable instructions for use in implementing dPCR systems and methods in accordance with one or more embodiments reside in computer program product 111 which is stored on one or more computers 107 in storage 108 and those instructions are executable by processor 110. When processor 110 is executing the instructions of computer program product 111, the instructions, or a portion thereof, are typically loaded into working memory 109 from which the instructions are readily accessed by processor 110. In the illustrated embodiment, computer program product 104 is stored in storage 108 or another non-transitory computer readable medium (which may include being distributed across media on different devices and different locations). In alternative embodiments, the storage medium is transitory.

In one embodiment, processor 110 in fact comprises multiple processors which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 110 may be implemented as one or more general purpose processors (preferably having multiple cores) without necessarily departing from the spirit and scope of the present invention.

User device 112 incudes a display 113 for displaying results of processing carried out by one or more computers 107 executing instructions of computer program product 111. In some embodiments, processing supporting analysis performed by system 1000, or a portion thereof, may be executed by one or more processors residing on PCR instruments 101 and 102 and/or user device 112 and all or portions of associated computer-executable instructions may be stored in computer readable storage at PCR instruments 101 and/or 102 or at user device 112. Such alternatives do not depart from the scope of the invention.

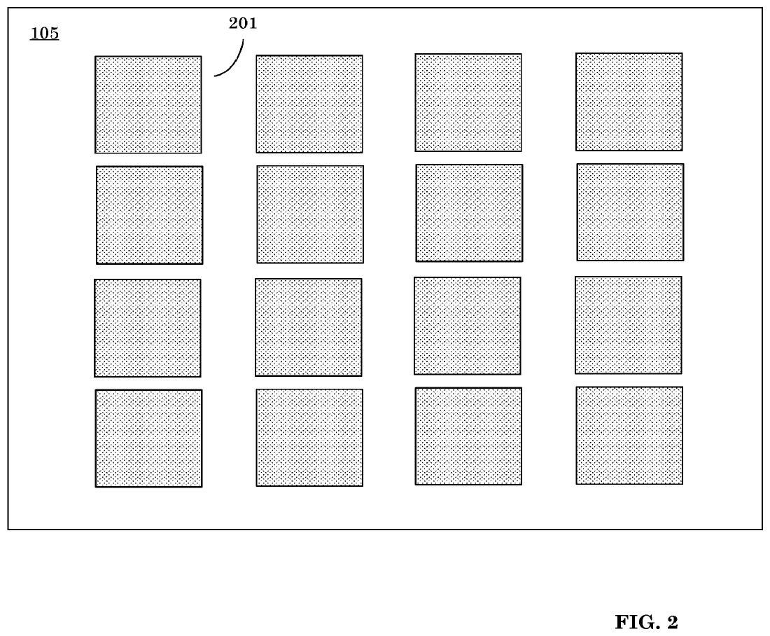

FIG. 2 illustrates further details of sample plate 105 referenced in the context of FIG. 1. In this example, sample plate 105 includes arrays 201 of sample partitions (sample partitions not separately shown). In the illustrated embodiment, each array 201 includes over 20,000 individual partitions (specifically, 20,480 partitions in one example) and a sample plate 105, as illustrated, includes 16 such arrays 201. In existing methods, a sample loaded into an array 201 of sample plate 105 is analyzed as a single unit and thresholds for dPCR analysis are determined for each color channel using only fluorescence data of partitions in the same array 201.

However, embodiments of the present disclosure facilitate larger scale and more flexible sample grouping. As will be explained in further detail herein, embodiments of the disclosure allow grouping samples together at different levels of aggregation including across a single array, across multiple arrays on a single sample plate, across multiple arrays on different sample plates processed by the same instrument, and also across multiple arrays on different sample plates processed by different instruments.

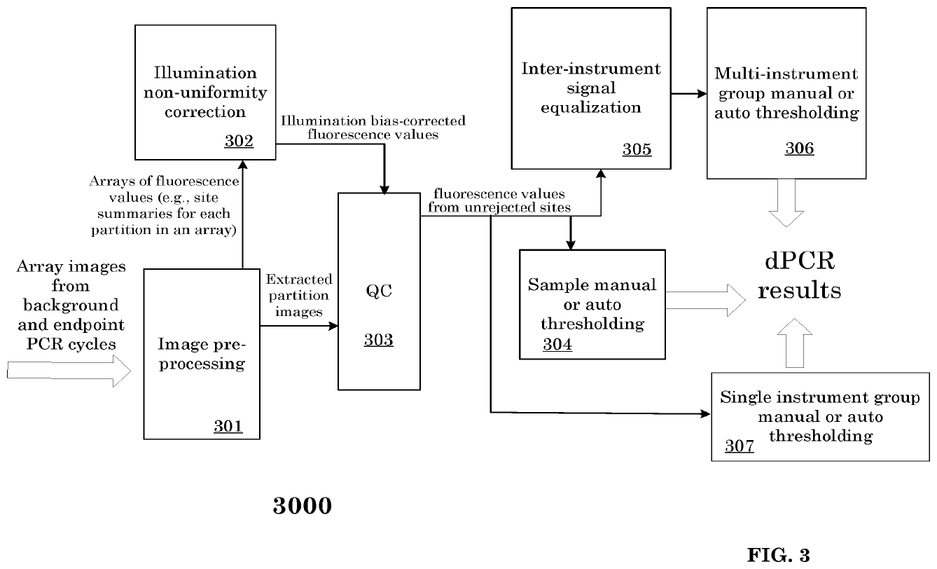

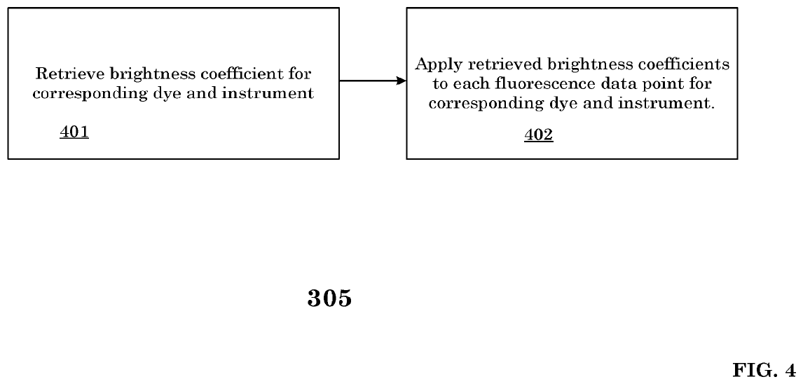

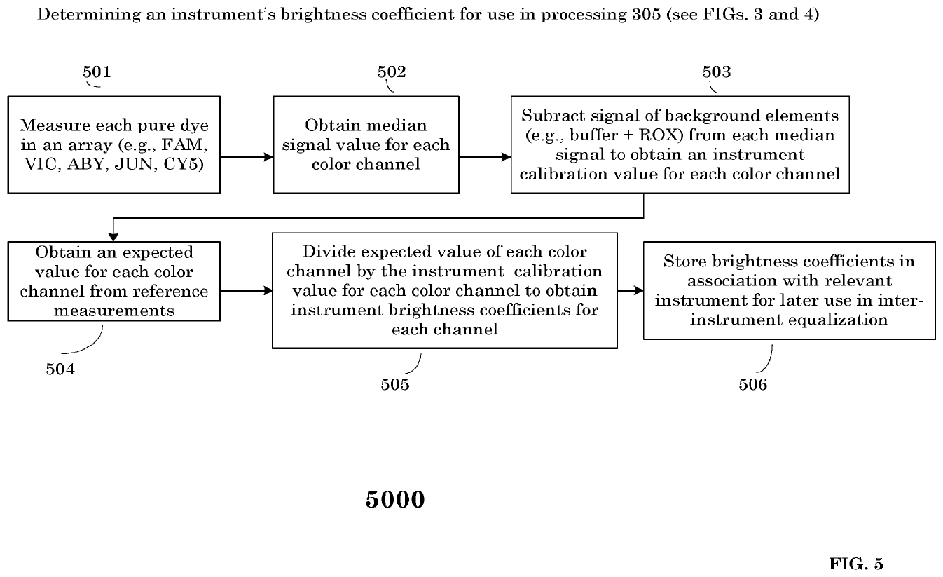

FIG. 3 illustrates a high-level block diagram of processing 3000 carried out by a dPCR analysis system in accordance with some embodiments of the present disclosure. Image pre-processing block 301 receives images of arrays of partitions (e.g., arrays 201 illustrated in FIG. 2). In one embodiment, image pre-processing block 301 receives images captured by a PCR instrument at (just after) a background cycle and at (just after) an endpoint cycle of a PCR assay. A background cycle corresponds to a cycle number low enough (for example, cycle 15) that amplification would not be expected to have occurred. An endpoint cycle corresponds to a cycle number high enough (for example, cycle 40) that amplification would be expected to have occurred in partitions in which a target analyte was present. Pre-processing block 301 processes the received images to provide extracted partition images to quality control (QC) block 303 and to provide arrays of fluorescence values to illumination non-uniformity correction block 302.

In an embodiment, each fluorescence value in an array of fluorescence values is a value that summarizes pixel intensity values for a given color channel for all pixels corresponding to a given (one) sample partition site (e.g., a microchamber). For example, a given microchamber in an array of microchambers might correspond to 256 pixels of the larger image of the array.

Either the median or the average of those 256 pixel intensity values can be used as a summary value for the given microchamber. In one embodiment, the median is used. The summary values for all the microchambers in an array collectively form what may be called a “pseudo” image in that they are not for displaying an image of the array, but do represent relevant fluorescence (illumination) information obtained from the digital image and are usable in PCR analysis including in dPCR analysis.

Image pre-processing block 301 also provides extracted partition images (each including pixel values for all 256 pixels corresponding to a given partition) to machine-learning based quality control (QC) processing block 303. In other words, image pre-processing block 301 extracts, from the whole image of an array, sets of pixels corresponding to individual microchambers in the array. QC processing block 303 analyzes the partition images and determines whether individual partitions in an array (or in a sample group) exhibit QC issues such that the partition should either be excluded from downstream dPCR analysis or should be flagged with a QC warning to be considered by a user and/or by automated downstream processing. Examples of systems and methods that can be used to implement the QC processing of block 303 are more fully described in Applicant's co-pending application filed on the same date as the present application and identified by attorney docket number 10121-3009000TP386129USPRV1. The contents of that application are hereby incorporated by reference in their entirety.

Illumination non-uniformity correction 302 block processes fluorescence values corresponding to an array to apply a correction for illumination bias that might exist associated with a particular dye channel of a particular PCR instrument. Examples of systems and methods that can be used to implement the illumination processing of block 302 are more fully described in Applicant's co-pending application filed on the same date as the present application and identified by attorney docket number 10121-3008900TP386033USPRV1. The contents of that application are hereby incorporated by reference in their entirety.

In the illustrated embodiment, QC processing block 303 also receives (in addition to extracted partition images) bias-corrected fluorescence values from block 302. For some QC processing tasks, block 303 uses these site summary values (including a summarized value for each partition). For other QC processing tasks, block 303 uses the extracted partition images (including separate values for each pixel of a partition).

Bias-corrected fluorescence values for sites that are not rejected by QC block 303 are further processed to set thresholds for each channel of each sample or sample group. The resulting thresholds are then used to determine corresponding dPCR quantification results.

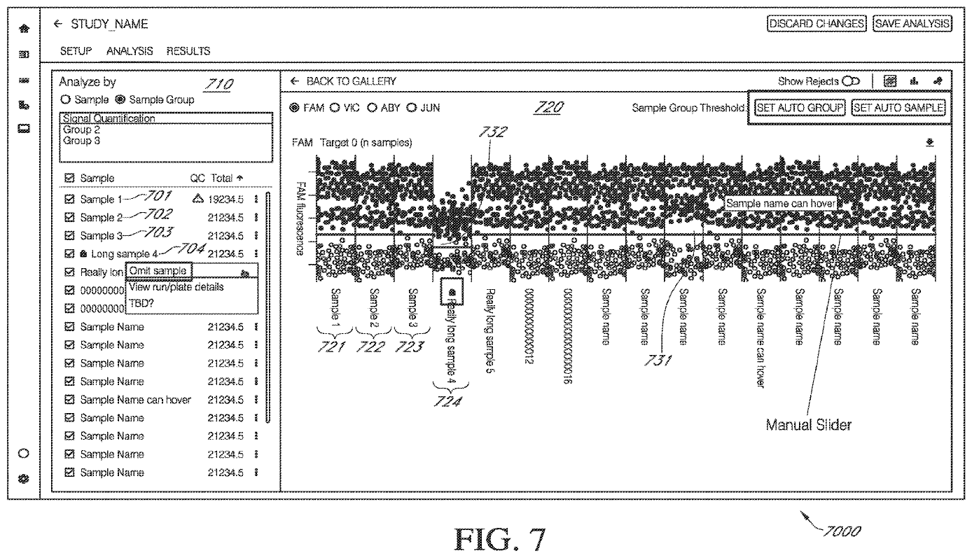

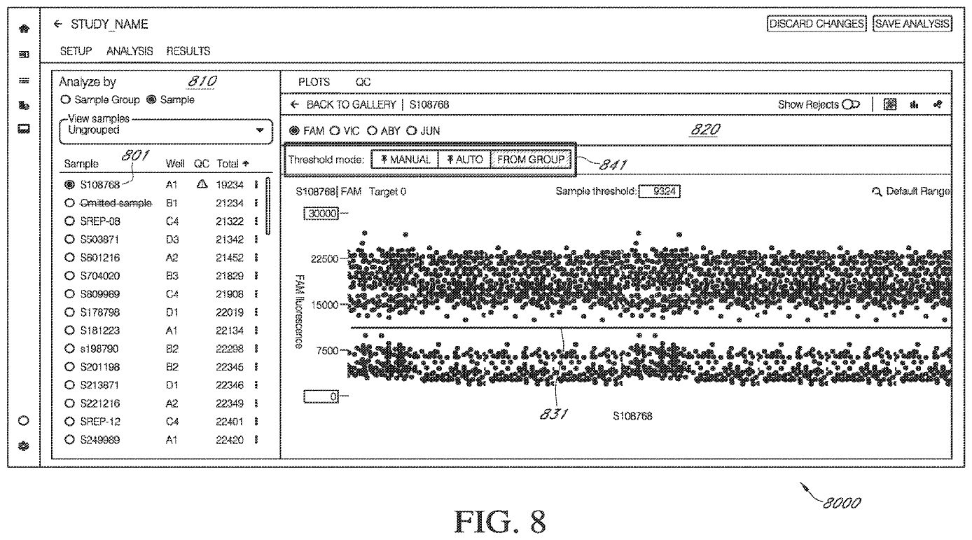

Notably, embodiments of the present disclosure provide a dPCR analysis system with the flexibility to set thresholds based on analysis of values from individual sample units (which generally correspond to values from a particular array 201) or from sample groups spanning multiple arrays (which may or may not be processed on the same PCR instrument). In one example, user input received from a graphical user interface (GUI) (such as, for example, shown in FIGS. 7 and 8 and further described below) is used to determine how data will be aggregated for a given channel for thresholding. Specifically, it is used to determine what aggregation of data will be processed together for thresholding purposes, for example, data from an individual sample unit (e.g., data from a single array 201), from a sample group that includes samples processed on the same PCR instrument (e.g., from multiple arrays 201 on one or more sample plates 105 processed by one PCR instrument), or from a sample group that includes some samples processed by a first PCR instrument and some sample processed by a second PCR instrument.

In the illustrated embodiment, inter-instrument signal equalization block 305 applies brightness coefficients determined for each dye channel of each instrument. This allows multi-instrument thresholding block 306 to process data from different PCR instruments together for thresholding purposes. Block 304 performs threshold processing on data that is not aggregated across different sample units. Block 307 performs threshold processing on data that is aggregated into sample groups including data across different sample units (i.e., different arrays of partitions) but not across different instruments.

In some embodiments, inter-instrument equalization processing 305 is performed on all data processed by blocks 304, 306 and/or 307, whether or not it is ultimately aggregated into sample groups including samples processed on different instruments. However, in some embodiments, for data that is being processed for only for a single sample unit, or for a sample group processed by the same instrument, the data can be analyzed using block 304 or block 307 without necessarily performing the processing of inter-instrument signal equalization block 305. For manual thresholding, whether performed on data across sample groups or on data from a single sample, a data plot is electronically displayed on a GUI (examples of which are illustrated in FIGS. 7 and 8) and a user can manually set a threshold based on visual inspection of the data. For auto-thresholding, one or more computer-executed algorithms are performed to determine an optimal threshold automatically. Various techniques for auto-thresholding may be used. Some examples of systems and methods that can be used to implement auto-thresholding (whether done on data from a single sample unit or on a sample group spanning multiple sample units) are more fully described in Applicant's co-pending application filed on the same date as the present application and identified by attorney docket no. 10121-3008800 TP385920USPRV1. The contents of that application are hereby incorporated by reference in their entirety.

FIG. 4 is a flow diagram showing the processing steps of inter-instrument equalization processing 305. Step 401 retrieves brightness coefficients corresponding to each dye and instrument. In one example, for a given instrument, if there are 5 different dye channels processed on that instrument, then 5 different brightness coefficients have been determined for that instrument (one for each dye channel). In some embodiments, the dye coefficients for color channels for a given instrument are stored electronically on the instrument and retrieved by the dPCR analysis system when needed. In other embodiments, they are stored in other electronic storage accessible by the dPCR analysis system implementing equalization processing 305. Step 402 applies the retrieved brightness coefficients to current fluorescent data corresponding to samples processed by the dye channels and instruments corresponding to the retrieved brightness coefficients.

FIG. 5 is a flow diagram illustrating a method 5000 for determining the brightness coefficients retrieved and applied by processing 305 of FIG. 4. The method is in accordance with one embodiment of the disclosure. The illustrated example will be described with respect to determining brightness coefficients for each color channel of one instrument. In a particular implementation, the method would be repeated for each instrument of the corresponding multi-instrument dPCR system.

Step 501 measures fluorescent signals from calibration runs for each pure dye processed by the PCR instrument. Step 502 obtains a median signal value for each dye channel. Step 503 subtracts, from each median signal, a portion of the signal corresponding to background elements. In one example, a background signal is obtained by measuring a reference solution including only background elements of a PCR master mix. For example, such a reference solution might only include buffer and ROX, a type of inert dye whose fluorescent signal is independent of amplification.

Step 504 obtains an expected value for each color channel from reference measurements. In one example, reference measurements are obtained by performing several calibration measurements on different PCR instruments and then summarizing them (e.g., taking median or mean) to obtain an expected value for each color channel. In the example of a two-instrument dPCR system (e.g., instruments 101 and 102 of system 1000 shown in FIG. 1), the expected values may be obtained using different instruments than either instrument of the two instrument system. For example, three different instruments may be used to perform several calibration measurements and the average or median values obtained from those measurements in each channel may be taken as an “expected” value for a given color channel. However, in an alternative embodiment, one or a combination of two or more instruments of a multiple instrument system might be used to provide the “expected” values without relying on measurements from instruments other than the instruments of the given multi-instrument system.

Step 505 divides the expected value (obtained from one or more other instruments) and the calibration value obtained from the given instrument to obtain a brightness coefficient for each color channel of the given instrument. Step 506 stores the brightness coefficients in association with the relevant instrument (and relevant color channel) for later use in inter-instrument equalization (e.g., as carried out by processing 305 shown in FIG. 4).

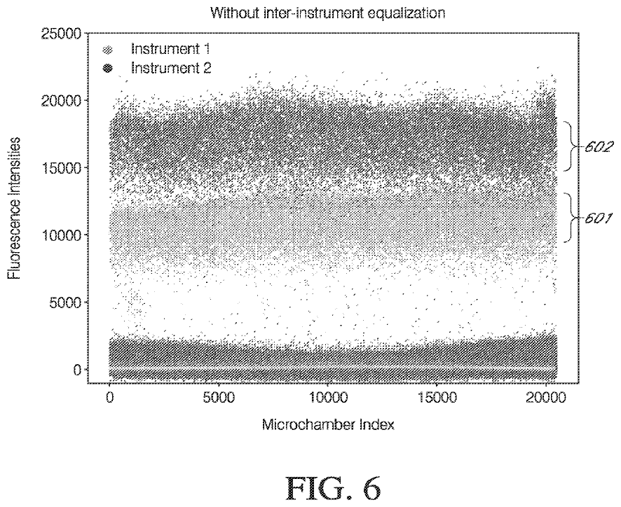

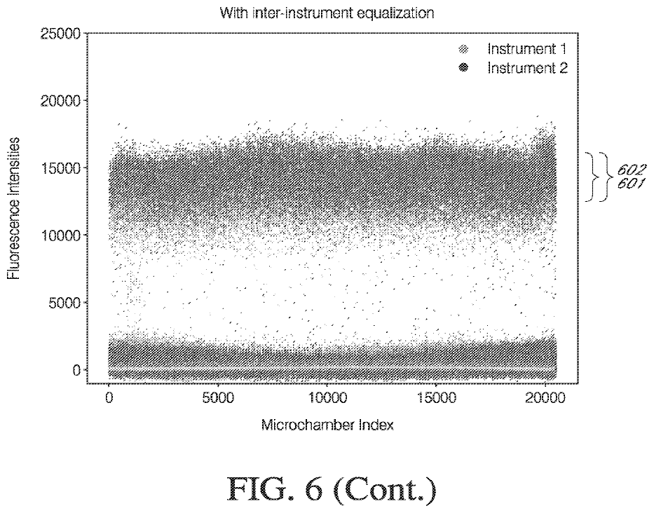

FIGS. 6A and 6B illustrate how inter-instrument equalization, as performed by processing 305 using brightness coefficients obtained by method 5000, can enable and/or improve group thresholding by minimizing differences between signals obtained from different instruments.

FIG. 6A illustrates data obtained from a first instrument, “Instrument 1” (e.g., instrument 101 of system 1000 shown in FIG. 1) and a second instrument “Instrument 2” (e.g., instrument 102 of system 1000 shown in FIG. 1). The data shows summarized fluorescent values for each partition of over 20,000 partitions, for a single color channel. The data points shown in light grey were obtained from processing by first PCR instrument, “Instrument 1”. The data points shown in dark grey were obtained from processing by a second PCR instrument, “Instrument 2.”

As illustrated in FIG. 6A, the data shows a positive band 601 for Instrument 1 that is distinctly different than positive band 602 for Instrument 2. Such differences across data obtained from two-different instruments can result in difficulty and errors in identifying an appropriate group threshold and can result in errors in the resulting positive/negative dPCR determinations that are based on that threshold. Inter-instrument equalization processing, in accordance with some embodiments of the present disclosure, has not been applied to the data illustrated in FIG. 6A.

FIG. 6B illustrates the effect of inter-instrument equalization in accordance with some embodiments of the present disclosure. Specifically, in FIG. 6B, positive band 601 corresponding to data from Instrument 1 significantly overlaps with positive band 602 corresponding to data obtained from Instrument 2 such that the data can be readily combined for purposes of group thresholding.

FIGS. 6A and 6B are both generated using the same fluorescent data from each instrument. However, inter-instrument signal equalization in accordance with some embodiments of the present disclosure has been applied to the data in FIG. 6B but not to the data in FIG. 6A.

FIG. 7 illustrate a graphical user interface (GUI) 7000 implemented by a system such as system 1000 of FIG. 1 in accordance with some embodiments of the disclosure. As shown, GUI 7000 allows flexible grouping and thresholding of samples for analysis. GUI 700 includes windows 710 and 720. In window 710, a user can select each sample to be included in the data display of window 720. As illustrated, all samples shown in the relevant sample group have been selected (as represented by the “checked” boxes). For example, samples 701, 702, 703, and 704, as well as the other samples shown have been selected. Also, as shown in window 710, a sample within the analyzed group can be “locked” from group thresholding with respect to the displayed group. In this example, sample 704 has been locked so that a separate threshold can be set based on analysis of just that sample's data rather than based on the data from all samples in the group.

Data display window 720 includes separate regions along the horizontal axis corresponding to each sample's data. For example, regions 721, 722, 723, and 724 are used to display data corresponding to, respectively, samples 701, 702, 703, and 704.

In this example, two different thresholds have been determined. A determined group threshold is represented by line graphic 731. A determined sample threshold, applying only to locked sample 704 (corresponding to the data displayed in region 724), is represented by line graphic 732. In the upper right corner of window 720, the “SET AUTO GROUP” and “SET AUTO SAMPLE” options have been selected. This means that thresholds will be set automatically via computerized analysis of the data. In the case of the group threshold shown by line 731, the threshold has been determined by analyzing all the data except for the data corresponding to sample 704 (displayed in region 724). In the case of the sample threshold shown by line 732, the threshold has been determined by analyzing just the data corresponding to sample 704 (shown in region 724). However, in this example, a user can also set the group and/or the manual thresholds manually by, for example, manually moving lines 731 and/or 732 via GUI 7000.

FIG. 8 illustrate a GUI 8000 implemented by a system such as system 1000 of FIG. 1 in accordance with some embodiments of the disclosure. GUI 8000 includes windows 810 and 820. In this example, the user has interacted with window 810 to select to view and analyze “per sample” rather than across a sample group and has selected a sample 801 for analysis. Thus, only data for one sample, i.e., sample 801, is displayed in window 820. GUI 8000 included, in window 820, threshold mode selector 841. This allows a user to select the manner in which a threshold, represented by line graphic 831, is determined. In this example, the user has used threshold mode selector 841 to select “FROM GROUP” which results in use of a threshold that has been determined from the larger sample group of which sample 801 is a part. However, the user could also, alternatively, select “MANUAL” to select the threshold manually, or “AUTO” to have the system automatically determine a threshold based on analysis of the selected sample's data.

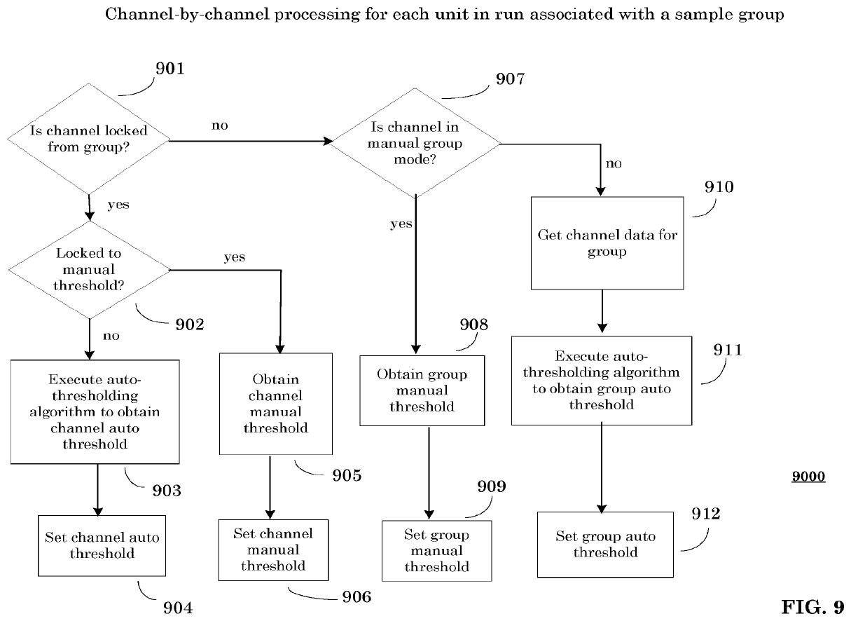

FIG. 9 illustrates processing 9000 used to determine a threshold to set for display in a GUI such as, for example GUI 7000 or GUI 8000.

Processing 9000 shows channel-by-channel processing for each unit in a sample group for samples that have been processed for dPCR analysis.

Step 901 determines if the current channel is locked from the group (i.e., that channel's data for a given sample would not be considered as part of the sample group thresholding determination). If the result of step 901 is yes, then step 902 determines if it is locked to manual thresholding (i.e., that the user has opted to set the sample's threshold for that channel manually rather than by computerized auto-thresholding). If the result of step 902 is yes, then step 905 obtains a channel manual threshold and step 906 sets that as the threshold. If the result of step 902 is no, then step 903 executes an auto-thresholding algorithm (such as, for example, as disclosed in co-pending application filed on the same date as the present application and identified by attorney docket no. 10121-3008800 TP385920USPRV1) to obtain an auto threshold using that sample's data and step 904 sets the threshold to the threshold determined by the auto-thresholding algorithm.

If the result of step 901 is no, then step 907 determines whether the current channel is in manual group mode. If the result of step 907 is yes, then step 908 obtains a group manual threshold and step 909 sets that as the threshold.

If the result of step 907 is no, then step 910 retrieves channel data for all samples in the group. Step 911 then executes an auto-threshold algorithm (such as, for example, as disclosed in applicant's co-pending application referenced above) and step 912 sets that as a threshold.

In one aspect, embodiments of the present disclosure improved the throughput, accuracy, and/or flexibility of dPCR technology. For example, allowing samples to be grouped for thresholding purposes can, in some instances, lead to more accurate thresholding which in turn, can lead to more accurate qPCR results. Allowing samples to be grouped can enable pooled analysis which may be used to quantify all the samples as one large logical entity. Sample grouping can also enable a large number of samples to be treated as replicates to improve statistical confidence in the quantification results.

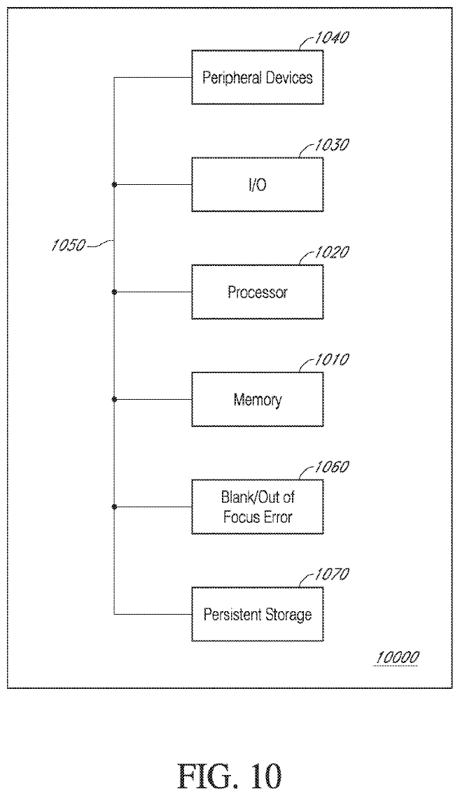

FIG. 10 illustrates an exemplary computer system configurable by a computer program product to carry out one or more of the components of a dPCR test and analysis system and associated interactive graphical user interface consistent with embodiments of the present disclosure. Computer system 10000 executes instruction code contained in a computer program product 1060. Computer program product 1060 comprises executable code in an electronically readable medium that may instruct one or more computers such as computer system 1000 to perform processing that accomplishes the exemplary method steps performed by the embodiments referenced herein. The electronically readable medium may be any non-transitory medium that stores information electronically and may be accessed locally or remotely, for example, via a network connection. In alternative embodiments, the medium may be transitory. The medium may include a plurality of geographically dispersed media, each configured to store different parts of the executable code at different locations or at different times. The executable instruction code in an electronically readable medium directs the illustrated computer system 10000 to carry out various exemplary tasks described herein. The executable code for directing the carrying out of tasks described herein would be typically realized in software. However, it will be appreciated by those skilled in the art that computers or other electronic devices might utilize code realized in hardware to perform many or all the identified tasks without departing from the present disclosure. Those skilled in the art will understand that many variations on executable code may be found that implement exemplary methods within the spirit and the scope of the present disclosure.

The code or a copy of the code contained in computer program product 1460 may reside in one or more storage persistent media (not separately shown) communicatively coupled to computer system 10000 for loading and storage in persistent storage device 1070 and/or memory 1010 for execution by processor 1020. Computer system 10000 also includes I/O subsystem 1030 and peripheral devices 1040. I/O subsystem 1030, peripheral devices 1040, processor 1020, memory 1010, and persistent storage device 1070 are coupled via bus 1050.

Like persistent storage device 1070 and any other persistent storage that might contain computer program product 1060, memory 1010 is a non-transitory media (even if implemented as a typical volatile computer memory device). Moreover, those skilled in the art will appreciate that in addition to storing computer program product 1060 for carrying out the processing described herein, memory 1010 and/or persistent storage device 1070 may be configured to store the various data elements referenced and illustrated herein.

Those skilled in the art will appreciate computer system 10000 illustrates just one example of a system in which a computer program product in accordance with an embodiment of the present disclosure may be implemented. To cite but one example of an alternative embodiment, storage and execution of instructions contained in a computer program product in accordance with an embodiment of the present disclosure may be distributed over multiple computers, such as, for example, over the computers of a distributed computing network. In the context of the specification, at least the following embodiments are described.

Embodiment 1 is a digital polymerase chain reaction (dPCR) system comprising two or more dPCR instruments; one or more data processors communicatively coupled to the two or more dPCR instruments; and one or more computer readable media communicatively coupled to the one or more processors storing executable instructions that, when executed by the one or more data processors, perform processing comprising receiving fluorescence data corresponding to processing of partitioned samples on sample plates processed by the two or more dPCR instruments; and applying respective brightness coefficients to respective portions of the fluorescence data, a respective brightness coefficient corresponding to a respective color channel of a respective one of the two or more dPCR instruments that generated corresponding respective portions of the fluorescence data; wherein applying the respective brightness coefficients minimizes instrument-related differences in brightness for a respective color channel such that fluorescent data from samples processed on different ones of the two or more dPCR instruments can be grouped together in a sample group for purposes determining dPCR results corresponding to the sample group using a group threshold.

Embodiment 2 is the dPCR system of embodiment 1 wherein the respective brightness coefficients for respective color channels of a first instrument of the two or more instruments are obtained using calibration fluorescent values obtained from one of more calibration runs of the first instrument and using expected fluorescent values obtained from one or more calibration runs of one or more reference instruments. Embodiment 3 is the dPCR system of embodiment 2 wherein the one or more reference instruments do not include any of the two or more dPCR instruments of the dPCR system. Embodiment 4 is the dPCR system of embodiment 2 wherein the one or more reference instruments do include at least one of the two or more dPCR instruments of the dPCR system. Embodiment 5 is the dPCR system of any one of embodiments 2-4 wherein a respective calibration fluorescent value for a respective color channel comprises a respective median value of values obtained from measuring fluorescence of partitions of a calibration plate corresponding to the respective color channel. Embodiment 6 is the dPCR system of any one of embodiments 2-5 wherein calibration values and expected fluorescence values have been adjusted by removing signal portions corresponding to background elements of one or more calibration runs from which the calibration values and expected fluorescence values are obtained. Embodiment 7 is the dPCR system of embodiment 6 wherein background elements comprise a buffer portion of a calibration solution. Embodiment 8 is the dPCR system of any of embodiments 6-7 wherein background elements comprise a ROX dye portion of a calibration solution.

Embodiment 9 is a method of quantifying target analytes in a plurality of biological samples using digital polymerase chain reaction (dPCR), the method comprising subjecting a plurality of biological samples to a dPCR assay using one or more dPCR instruments, each sample of the plurality of samples being partitioned into an array of sample partitions on a sample plate comprising a plurality of arrays of partitions and the plurality of samples being arranged across a plurality of sample plates processed by the one or more DPCR instruments; receiving, at one or more computers, fluorescence data corresponding to the plurality of samples, the fluorescence data including respective fluorescence values for corresponding respective ones of a plurality of sample partitions; executing processing using one or more processors of the one or more computers, the processing comprising providing a graphical user interface GUI on an electronic display of a user device, the user device being configured to receive user input to define sample groups corresponding to the plurality of samples; enabling a user to select a plurality of samples, via the GUI, to be included in a sample group wherein the plurality of samples selected for the sample group comprise at least one sample processed on a first sample plate and at least one sample processed on a second sample plate different from the first sample plate; and obtaining a threshold value for identifying fluorescent values representing amplification at an individual sample partition, wherein the obtained threshold value is usable for dPCR analysis of all samples in the sample group that are selected by the user via the GUI for group thresholding.

Embodiment 10 is the method of embodiment 9 wherein a sample plate comprises a plurality of arrays of partitions, each array of partitions comprising at least 8,000 partitions. Embodiment 11 is the method of any one of embodiments 9 and 10 wherein the first sample plate is processed by a first dPCR instrument and the second sample plate is processed by a second dPCR instrument. Embodiment 12 is the method of embodiment 11 wherein respective first brightness coefficients corresponding to respective dye channels of the first instrument are applied to fluorescent data corresponding to samples processed on the first sample plate and respective second brightness coefficients corresponding to respective dye channels of the second instrument are applied to fluorescent data corresponding to samples processed on the second sample plate such that fluorescent data corresponding to samples processed on the first sample plate can be analyzed together with fluorescent data corresponding to samples processed on the second sample plate. Embodiment 13 is the method of any one of embodiments 9-12 wherein: the GUI enables a user to select between using a threshold obtained from automated computerized analysis of fluorescent data corresponding to the sample group and using a threshold manually obtained from user interaction with the GUI via operation of the user device. Embodiment 14 is the method of any one of embodiments 9-13 wherein the GUI enables a user to exclude fluorescent data for any one of the samples from group thresholding for a particular channel. Embodiment 15 is the method of any one of embodiments 9-14 wherein the GUI allows a user to exclude fluorescent data for any one of the samples from auto thresholding for a particular channel.

Embodiment 16 is a computer program product comprising processor-executable instructions stored in a non-transitory computer readable medium that, when executed by one or more processors, cause the one or more processors to perform processing comprising receiving fluorescence data corresponding to a plurality of samples subjected to a d digital polymerase chain reaction (dPCR) assay, the fluorescence data including respective fluorescence values for corresponding respective ones of a plurality of sample partitions; providing a graphical user interface GUI on an electronic display of a user device, the user device being configured to receive user input to define sample groups corresponding to the plurality of samples; enabling a user to select a plurality of samples, via the GUI, to be included in a sample group wherein the plurality of samples selected for the sample group comprise at least one sample processed on a first sample plate and at least one sample processed on a second sample plate different from the first sample plate; and obtaining a threshold value for identifying fluorescent values representing amplification at an individual sample partition, wherein the obtained threshold value is usable for dPCR analysis of all samples in the sample group that are selected by the user via the GUI for group thresholding.

Embodiment 17 is the computer program product of embodiment 16 wherein the first sample plate is processed by a first dPCR instrument and the second sample plate is processed by a second dPCR instrument. Embodiment 18 is the computer program product of embodiment 17 wherein the processing further comprises applying respective first brightness coefficients corresponding to respective dye channels of the first instrument to fluorescent data corresponding to samples processed on the first sample plate and applying respective second brightness coefficients corresponding to respective dye channels of the second instrument to fluorescent data corresponding to samples processed on the second sample plate such that fluorescent data corresponding to samples processed on the first sample plate can be analyzed together with fluorescent data corresponding to samples processed on the second sample plate.

Embodiment 19 is the computer program product of any one of embodiments 16-18 wherein the GUI enables a user to select between using a threshold obtained from automated computerized analysis of fluorescent data corresponding to the sample group and using a threshold manually obtained from user interaction with the GUI via operation of the user device. Embodiment 20 is the computer program product of any one of embodiments 16-19 wherein the GUI enables a user to exclude fluorescent data for any one of the samples from group thresholding for a particular channel. Embodiment 21 is the computer program product of any one of embodiments 16-20 wherein the GUI allows a user to exclude fluorescent data for any of the samples from auto thresholding for a particular channel.

1. A digital polymerase chain reaction (dPCR) system comprising:
two or more dPCR instruments;
one or more data processors communicatively coupled to the two or more dPCR instruments; and
one or more computer readable media communicatively coupled to the one or more processors storing executable instructions that, when executed by the one or more data processors, perform processing comprising:
receiving fluorescence data corresponding to processing of partitioned samples on sample plates processed by the two or more dPCR instruments; and
applying respective brightness coefficients to respective portions of the fluorescence data, a respective brightness coefficient corresponding to a respective color channel of a respective one of the two or more dPCR instruments that generated corresponding respective portions of the fluorescence data;
wherein applying the respective brightness coefficients minimizes instrument-related differences in brightness for a respective color channel such that fluorescent data from samples processed on different ones of the two or more dPCR instruments can be grouped together in a sample group for purposes determining dPCR results corresponding to the sample group using a group threshold.

2. The dPCR system of claim 1 wherein the respective brightness coefficients for respective color channels of a first instrument of the two or more instruments are obtained using calibration fluorescent values obtained from one of more calibration runs of the first instrument and using expected fluorescent values obtained from one or more calibration runs of one or more reference instruments.

3. The dPCR system of claim 2 wherein the one or more reference instruments do not include any of the two or more dPCR instruments of the dPCR system.

4. The dPCR system of claim 2 wherein the one or more reference instrument do include at least one of the two or more dPCR instruments of the dPCR system.

5. The dPCR system of claim 2 wherein a respective calibration fluorescent value for a respective color channel comprises a respective median value of values obtained from measuring fluorescence of partitions of a calibration plate corresponding to the respective color channel.

6. The dPCR system of claim 2 wherein calibration values and expected fluorescence values have been adjusted by removing signal portions corresponding to background elements of one or more calibration runs from which the calibration values and expected fluorescence values are obtained.

7. The dPCR system of claim 6 wherein background elements comprise a buffer portion of a calibration solution.

8. The dPCR system of claim 6 wherein background elements comprise a ROX dye portion of a calibration solution.

9. A method of quantifying target analytes in a plurality of biological samples using digital polymerase chain reaction (dPCR), the method comprising:
subjecting a plurality of biological samples to a dPCR assay using one or more dPCR instruments, each sample of the plurality of samples being partitioned into an array of sample partitions on a sample plate comprising a plurality of arrays of partitions and the plurality of samples being arranged across a plurality of sample plates processed by the one or more DPCR instruments;
receiving, at one or more computers, fluorescence data corresponding to the plurality of samples, the fluorescence data including respective fluorescence values for corresponding respective ones of a plurality of sample partitions executing processing using one or more processors of the one or more computers, the processing comprising:
providing a graphical user interface GUI on an electronic display of a user device, the user device being configured to receive user input to define sample groups corresponding to the plurality of samples;
enabling a user to select a plurality of samples, via the GUI, to be included in a sample group wherein the plurality of samples selected for the sample group comprise at least one sample processed on a first sample plate and at least one sample processed on a second sample plate different from the first sample plate; and
obtaining a threshold value for identifying fluorescent values representing amplification at an individual sample partition, wherein the obtained threshold value is usable for dPCR analysis of all samples in the sample group that are selected by the user via the GUI for group thresholding.

10. The method of claim 9 wherein a sample plate comprises a plurality of arrays of partitions, each array of partitions comprising at least 8,000 partitions.

11. The method of claim 9 wherein the first sample plate is processed by a first dPCR instrument and the second sample plate is processed by a second dPCR instrument.

12. The method of claim 11 wherein respective first brightness coefficients corresponding to respective dye channels of the first instrument are applied to fluorescent data corresponding to samples processed on the first sample plate and respective second brightness coefficients corresponding to respective dye channels of the second instrument are applied to fluorescent data corresponding to samples processed on the second sample plate such that fluorescent data corresponding to samples processed on the first sample plate can be analyzed together with fluorescent data corresponding to samples processed on the second sample plate.

13. The method of claim 9 wherein: the GUI enables a user to select between using a threshold obtained from automated computerized analysis of fluorescent data corresponding to the sample group and using a threshold manually obtained from user interaction with the GUI via operation of the user device.

14. The method of claim 9 wherein the GUI enables a user to exclude fluorescent data for any one of the samples from group thresholding for a particular channel.

15. The method of claim 9 wherein the GUI allows a user to exclude fluorescent data for any one of the samples from auto thresholding for a particular channel.

16. A computer program product comprising processor-executable instructions stored in a non-transitory computer readable medium that, when executed by one or more processors, cause the one or more processors to perform processing comprising:
receiving fluorescence data corresponding to a plurality of samples subjected to a d digital polymerase chain reaction (dPCR) assay, the fluorescence data including respective fluorescence values for corresponding respective ones of a plurality of sample partitions;
providing a graphical user interface GUI on an electronic display of a user device, the user device being configured to receive user input to define sample groups corresponding to the plurality of samples;
enabling a user to select a plurality of samples, via the GUI, to be included in a sample group wherein the plurality of samples selected for the sample group comprise at least one sample processed on a first sample plate and at least one sample processed on a second sample plate different from the first sample plate; and
obtaining a threshold value for identifying fluorescent values representing amplification at an individual sample partition, wherein the obtained threshold value is usable for dPCR analysis of all samples in the sample group that are selected by the user via the GUI for group thresholding.

17. The computer program product of claim 16 wherein the first sample plate is processed by a first dPCR instrument and the second sample plate is processed by a second dPCR instrument.

18. The computer program product of claim 17 wherein the processing further comprises applying respective first brightness coefficients corresponding to respective dye channels of the first instrument to fluorescent data corresponding to samples processed on the first sample plate and applying respective second brightness coefficients corresponding to respective dye channels of the second instrument to fluorescent data corresponding to samples processed on the second sample plate such that fluorescent data corresponding to samples processed on the first sample plate can be analyzed together with fluorescent data corresponding to samples processed on the second sample plate.

19. The computer program product of claim 16 wherein the GUI enables a user to select between using a threshold obtained from automated computerized analysis of fluorescent data corresponding to the sample group and using a threshold manually obtained from user interaction with the GUI via operation of the user device.

20. The computer program product of claim 16 wherein the GUI enables a user to exclude fluorescent data for any one of the samples from group thresholding for a particular channel.

21. The computer program product of claim 16 wherein the GUI allows a user to exclude fluorescent data for any of the samples from auto thresholding for a particular channel.
